(19) United States
(12) Patent Application Publication
(10) Pub. No.: US 2009/0326361 A1
(43) Pub. Date: Dec. 31, 2009

(54) Computer aided method and apparatus

(76) Inventors: Shi Wei, Beijing (CN); Xiang Deng, Beijing (CN); Guang Wei Du, Beijing (CN)

Correspondence Address: HARNESS, DICKEY & PIERCE, P.L.C., P.O.BOX 8910, RESTON, VA, 20195, US

(21) Appl. No.: 12/457,909

(22) Filed: Jun. 25, 2009

(30) Foreign Application Priority Data
Jun. 26, 2008 (CN) 200810126363.4

(51) Int. Cl. A61B 5/055; G06K 9/00

(52) U.S. Cl. 600/410; 382/131

(57) ABSTRACT

A computer aided method and apparatus are disclosed. In at least one embodiment, the method includes acquiring image data of a scanned location of a subject by performing a nuclear magnetic resonance scan thereon, and displaying via a user interface an image formed by the image data; determining particular feature information by using an image segmentation algorithm to the image, determining the position of an acupotome insertion point according to the particular feature information, and displaying the position of the acupotome insertion point on the image. In at least one embodiment, the present invention can provide to a doctor the image information formed after scanning the scanned location, determine the particular feature information by using the image segmentation algorithm, and determine the position of the acupotome insertion point according to the determined particular feature information, thus providing to the doctor objective evidence for a decision on an acupotome surgical operation plan, thereby improving the accuracy of an acupotome treatment.

### PRIORITY STATEMENT

The present application hereby claims priority under 35 U.S.C. §119 on Chinese patent application number CN 200810126363.4 filed Jun. 26, 2008, the entire contents of which are hereby incorporated herein by reference.

### TECHNICAL FIELD

at least one embodiment of the present invention generally relates to computer aided technology, and particularly to a computer aided method and/or apparatus directed to acupotome treatment.

### BACKGROUND ART

The acupotome treatment, a new diagnosis and treatment technique in traditional Chinese medicine, was developed on the basis of the acupuncture treatments of traditional Chinese medicine and knowledge of human anatomy. In the acupotome treatment, a doctor uses a surgical tool called acupotome to resect the soft tissue around an injury to ensure the normal dynamical equilibrium of the human body. Acupotome treatments are increasingly gaining attention due to their outstanding curative effects to injured parts on cervical vertebrae, lumbar vertebrae and other parts that are difficult to cure.

During the determination of a surgical operation plan for an acupotome treatment, it is quite crucial to determine the position of an acupotome insertion point. However, in the prior art, the position of an acupotome insertion point is mainly determined by palpating a subject's ailing part by a doctor, according to his or her personal experiences and the subject's response to the palpation. That is to say, in existing acupotome treatments, the doctor cannot obtain particular feature information regarding an ailing part, such as detailed location of the ailment, type of ailment, detailed injury parameters, and the like. For example, if a subject is suffering from a cervical ailment, and the doctor cannot obtain the information regarding the exact location of the injury in the cervical part, the exact type of the cervical ailment, and the degree of the injury and so on, the location of an acupotome insertion point can only be determined according to the external conditions at the cervical part and the subject's response, and such subjective factors will lead to inaccuracy of the treatment, and may even damage the blood vessels or nerves in some severe cases.

### SUMMARY

In view of the situation, at least one embodiment of the present invention provides a computer aided method and/or apparatus so as to improve the accuracy of an acupotome treatment.

In at least one embodiment, a computer aided method comprises:

A. acquiring image data of a scanned location of a subject by performing a nuclear magnetic resonance scan thereon, and displaying the image formed by said image data via a user interface; and

B. determining particular feature information by using an image segmentation algorithm to said image, determining the position of an acupotome insertion point according to said particular feature information, and displaying the position of said acupotome insertion point on said image.

In at least one embodiment, the determining of the particular feature information by using a segmentation algorithm to said image in step B comprises:

B1. processing said image by using the image segmentation algorithm, and displaying the result of said processing on said user interface; and

B2. searching for particular feature information corresponding to a processed result that is the closest to said processed result in a pre-set information database, wherein said information database stores particular feature information corresponding to a variety of processed results; or, providing to a user a measurement tool on said user interface, and determining the particular feature information according to a measured result obtained by the user after having performed a measurement with the measurement tool.

In at least one embodiment, the determining the position of an acupotome insertion point according to said particular feature information in step B comprises: upon receiving a trigger of determining an surgical operation plan performed by the user via the user interface, determining the position of the acupotome insertion point by using a pre-set strategy according to said particular feature information.

Said scanned location for nuclear magnetic resonance scanning is a location for scanning that is marked with a checkpoint in advance;

the position of the checkpoint is displayed in said image formed; and

before displaying the position of said acupotome insertion point on said image, it further comprises: judging whether the distance between the position of said checkpoint and the position of said acupotome insertion point is within a set range, if it is no, then continuing the execution of the operation of displaying the position of said acupotome insertion point on said image, and prompting the user that the marking of the position of the checkpoint is wrong; and if it is yes, then prompting the user that the marking of the position of the checkpoint is correct.

In at least one embodiment, the method further comprises: executing said step A again on said scanned location of said subject after having performed an acupotome operation on the subject; and

displaying simultaneously said image before the acupotome operation and said image after the acupotome operation via the user interface, evaluating the operation's effectiveness by using a comparison result inputted by a doctor, and displaying the evaluation result via said user interface; or, continuing the execution of step B after having executed said step A again on said scanned location of said subject, comparing the particular feature information obtained before the acupotome operation with the particular feature information obtained after the acupotome operation, evaluating the operation's effectiveness according to the comparison result, and displaying the evaluation result via said user interface.

In a database set up for said subject, in at least one embodiment, one of said image data, the particular feature information, the position of the acupotome insertion point and the evaluation result or any combination thereof is stored; or the particular surgical operation plan information determined by a user according to said particular feature information is stored in the database set up for said subject, wherein said detailed surgical operation plan information comprises: a direction of the acupotome insertion or a depth of the acupotome insertion.

In at least one embodiment, a computer aided apparatus comprises: a magnetic resonance scanner, an analyzing unit, a plan determining unit and a user interface; wherein

said magnetic resonance scanner is used for acquiring image data of a scanned location of a subject by performing a nuclear magnetic resonance scanning thereon;

the analyzing unit is used for determining the particular feature information by using an image segmentation algorithm to the image formed by said image data;

the plan determining unit is used for determining the position of an acupotome insertion point according to said particular feature information; and

said user interface is used for displaying the image formed by said image data, and displaying the position of said acupotome insertion point on said image.

In at least one embodiment, the analyzing unit comprises: an image processing sub-unit, a database storing sub-unit and an information determining sub-unit; wherein

said image processing sub-unit is used for processing said image by using an image segmentation algorithm, and providing said processed result to said user interface;

said database storing sub-unit is used for storing the pre-set particular feature information corresponding to a variety of processed results;

said information determining sub-unit is used for searching particular feature information corresponding to a processed result that is the closest to said processed result in said database storing sub-unit, and providing the particular feature information found to said user interface; and

said user interface is also used for displaying said processed result provided by said image processing sub-unit and said particular feature information provided by said information determining sub-unit.

In at least one embodiment, the analyzing unit comprises: an image processing sub-unit and an information determining sub-unit; wherein

said image processing sub-unit is used for processing said image by using an image segmentation algorithm, and providing said processed result to said user interface;

said user interface is also used for providing a measurement tool to a user, and providing the measured result obtained by the user after having performed a measurement with the measurement tool to said information determining sub-unit, and displaying said processed result provided by said image processing sub-unit and said particular feature information provided by said information determining sub-unit; and

said information determining sub-unit is used for determining the particular feature information according to said measured result, and providing said particular feature information to said user interface.

In at least one embodiment, the user interface is also used for sending a trigger notification to said plan determining unit upon receiving a trigger for determining a surgical operation plan performed by a user via the user interface; and

after having received said trigger notification, said plan determining unit determines the position of said acupotome insertion point by using a pre-set strategy according to said particular feature information.

In at least one embodiment, the scanned location by nuclear magnetic resonance scanning is a location for scanning that is marked with a checkpoint in advance; and the apparatus further comprises: a judging unit for judging whether the distance between the position of said checkpoint and the position of the acupotome insertion point determined by said plan determining unit is within a pre-set range, if it is no, then providing the position of the acupotome insertion point to said user interface for displaying, and prompting the user via the user interface that the marking of the position of the checkpoint is wrong; and if it is yes, then prompting the user via the user interface that the marking of the position of the checkpoint is correct.

In at least one embodiment, the apparatus further comprises: an effectiveness evaluating unit for performing evaluation of an operation's effectiveness according to the comparison result provided by said user interface, and providing the evaluation result to said user interface; and

said user interface is also used for displaying the image before the acupotome operation and the image after the acupotome operation which are acquired by said magnetic resonance scanner, receiving the comparison result inputted by a doctor, providing the comparison result to said effectiveness evaluating unit, and displaying said evaluation result.

In at least one embodiment, the apparatus further comprises: an effectiveness evaluating unit for comparing the feature information before the acupotome operation with the feature information after the acupotome operation determined by said analyzing unit, evaluating the operation's effectiveness according to said comparison result, and providing the evaluation result to said user interface; and

said user interface is also used for displaying said evaluation result.

In at least one embodiment, the apparatus further comprises: a database storing unit for storing one of said image data, the particular feature information, the position of the acupotome insertion point and the evaluation result or any combination thereof in a database set up for said subject;

said analyzing unit can also be used for acquiring the image data from said database storing unit; and

said plan determining unit and effectiveness evaluating unit are also used for acquiring the particular feature information from said database storing unit.

It can be seen from the above technical solutions that the method and apparatus provided in the embodiments of the present invention can acquire the image data of a scanned location by performing a nuclear magnetic resonance scanning on the subject's scanned location, and display the image formed by the image data via a user interface; determine particular feature information by using an image segmentation algorithm to said image, determine the position of an acupotome insertion point according to said particular feature information, and display the position of the acupotome insertion point on the image. Embodiments of the present invention can provide the image information formed after scanning the scanned location to a doctor, which image can reflect the ailment conditions of the scanned location, and further determine the particular feature information using the image segmentation algorithm, and can determine the position of the acupotome insertion point according to the determined particular feature information, which provide to the doctor objective evidence for determining an acupotome surgical operation plan, unlike in the prior art that an acupotome surgical operation plan is determined only according to some subjective evidence such as the subject's responses upon palpating and the doctor's personal experience, thereby improving the accuracy of the acupotome treatment.

### BRIEF DESCRIPTION OF THE ACCOMPANYING DRAWINGS

FIG. 1 is a flowchart of a main method provided according to embodiments of the present invention;

FIG. 2 is a flowchart of a particular method provided according to the embodiments of the present invention;

FIG. 3a is a structural diagram of an apparatus provided according to the embodiments of the present invention;

FIG. 3b is a structural diagram of another apparatus provided according to the embodiments of the present invention.

### DETAILED DESCRIPTION OF EMBODIMENTS OF THE INVENTION

In order to make the objects, technical solutions and advantages of embodiments of the present invention more apparent, the present invention will be described in detail in conjunction with the accompanying drawings and embodiments.

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

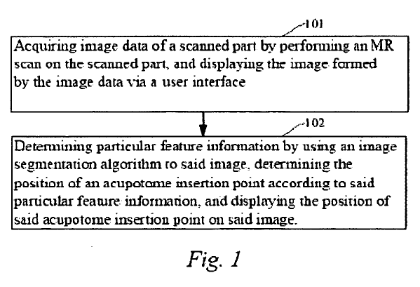

FIG. 1 is a flowchart of a main method provided according to an embodiment of the present invention, as shown in FIG. 1, the method mainly comprising the following steps:

step 101: acquiring image data of a scanned location by performing a nuclear magnetic resonance scanning on the scanned location, and displaying the image formed by the image data via a user interface; and

step 102: determining particular feature information with respect to said image by using an image segmentation algorithm, determining the position of an acupotome insertion point according to said particular feature information, and displaying the position of the acupotome insertion point on said image.

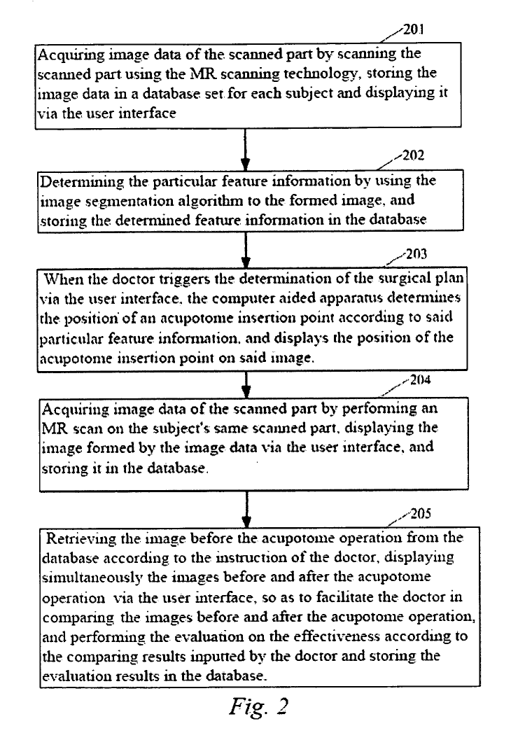

The above method will be described in conjunction with an embodiment below, FIG. 2 is a flowchart of the particular method provided according to the embodiment of the present invention, and as shown in FIG. 2, the method can comprise particularly the following step:

step 201: acquiring image data of the scanned location by scanning the scanned location using the nuclear magnetic resonance scanning technology, storing the image data in a database set up for each subject and displaying the data via the user interface.

In this step, the nuclear magnetic resonance scanning technology is introduced into the acupotome treatment, and assuming that the cervical part of a subject is injured, in this step, the computer aided apparatus can perform a nuclear magnetic resonance scanning on the subject's cervical part, so as to acquire image data of the cervical part, and display the nuclear magnetic resonance image of the cervical part obtained by scanning on the user interface.

In order to facilitate the management of the relevant diagnosis and treatment information of the subject, a database can be set up for the subject, the scanned image data of the scanned location is stored and an index can be created for the image data, for example, marking the information of the subject by the index, and it can further include, in the relevant diagnosis and treatment information of the subject, the scanning part information, scanning time information and so on, so as to facilitate the retrieval, comparison and analysis of the diagnosis and treatment information afterwards.

In addition, before performing the nuclear magnetic resonance scanning, the doctor can first mark a checkpoint on the scanned location of the subject and then carry out the scanning. The checkpoint can be marked by using vitamin E and so on, and the marked checkpoint will be shown clearly on the image after the nuclear magnetic resonance scanning is performed, so as to enable the doctor to quickly find on the image the region to be observed. In order to facilitate the doctor's observation, the doctor can determine a region concerned on the image via the user interface, and during a later diagnosis and treatment process notice can be paid only to the image within the region concerned.

Step 202: determining the particular feature information with respect to the formed image by using the image segmentation algorithm, and storing the determined feature information in the database.

In this step, the computer aided apparatus can employ an existing image segmentation algorithm to process the image formed and display the processed result on the user interface, and the computer aided apparatus can search, in a pre-stored information database in which the particular feature information corresponding to a variety of processed results are stored, the particular feature information corresponding to the processed result which is the closest, thereby determining the particular feature information. For example, a variety of treatment results and the corresponding particular feature information thereof can be stored in the information database in advance according to previous diagnosis results, and then comparing the image formed after a current scanning with a pre-stored ailment image, and the particular feature information corresponding to the closest ailment image is selected as the particular feature information of the current subject.

Alternatively, the computer aided apparatus can employ the existing image segmentation algorithm to process the formed image, display the processed result on the user interface, and then provide a measurement tool to the doctor via the user interface. The doctor uses the measurement tool on the user interface to measure the scanned location in the image, and the computer aided apparatus determines the particular feature information according to the measured result by the doctor. For example, a diameter measurement tool, a distance measurement tool, a color depth measurement tool and so on can be provided to the doctor via the user interface, and these measurement tools are used to perform the measurement of the diameter, location and color depth of a tumor, etc. on the image, and the computer aided apparatus can determine the particular feature information according to these measured results. The computer aided apparatus can also pre-store a variety of options of the particular feature information, and display these options to the doctor via the user interface, who can select directly the particular feature information of the subject in the options provided by the user interface, and then the computer aided apparatus stores the option selected by the user in a database and displays it to the doctor.

When displaying the image, 2D or 3D technology can be used to display the image of the subject according to the particular requirements of the doctor for diagnosis and treatment.

In this case, the process of the image segmentation algorithm processing belongs to the prior art and will not be discussed redundantly herein.

The particular feature information can be particular ailment position, type of ailment, injury parameters and so on, for example, after scanning a subject's cervical part and using the image segmentation algorithm to perform the processing, it is determined that the subject's ailment location is to the left side of the cervical vertebrae at a distance of 2 cm therefrom, the type of ailment is a tumor, the injury parameter is 2 cm in the tumor's diameter, the color depth is level 3, etc.

Step 203: when the doctor triggers the determination of a surgical operation plan on the user interface, the computer aided apparatus determines the position of an acupotome insertion point according to said particular feature information, and displays the position of the acupotome insertion point on said image.

In this step, when the doctor needs to determine the surgical operation plan of the acupotome operation, the computer aided apparatus can be triggered via the user interface to determine the surgical operation plan, and after the computer aided apparatus has been triggered, the position of the acupotome insertion point is determined according to the particular feature information determined in step 202 by using the pre-set strategy. For example, when the particular feature information is that the ailment location of the subject is to the left side of the cervical vertebrae at a distance of 2 cm-4 cm therefrom, the type of ailment is a tumor, the injury parameter is 2 cm in the tumor's diameter, the color depth is level 3, etc., the position of an acupotome insertion point can be determined as the middle of the tumor, that is, to the left side of the cervical vertebrae at a distance of 3 cm therefrom.

If the doctor marks a checkpoint on the scanned location in advance before performing a nuclear magnetic resonance scanning, then it can be judged whether the distance between the position of the marked checkpoint and the position of the determined insertion point is within a set range after having determined the position of the insertion point, if it is no, then displaying the position of determined acupotome insertion point on the image, and prompting the doctor that the marking of the position of the checkpoint is wrong; and if it is yes, then prompting the doctor that the marking of the position of the checkpoint is correct. The doctor can determine the general position of the acupotome insertion point in advance according to his or her own experiences, and mark it on the scanned location as a checkpoint, and then verify according to the position of the acupotome insertion point determined by the computer aided apparatus whether the position of the acupotome insertion point judged by himself or herself is accurate or not.

When the computer aided apparatus displays the position of the determined acupotome insertion point on the image, the doctor can determine other information of the acupotome surgical operation plan according to the particular feature information, for example, determine the insertion direction of the acupotome, the insertion depth of the acupotome and so on, that is to say, the computer aided apparatus provides objective evidence that determines the acupotome surgical operation plan for the doctor. The doctor can store the information of the determined acupotome surgical operation plan in a database via the user interface, so as to facilitate future retrieval and analysis.

The doctor can execute an acupotome operation on the scanned location of the subject according to the determined acupotome surgical operation plan, and after the acupotome operation, the following steps can be executed if the evaluation of the operation's effectiveness is needed:

step 204: acquiring image data of the scanned location by performing a nuclear magnetic resonance scanning on the subject's same scanned location, displaying the image formed by the image data via the user interface, and storing it in the database.

After the acupotome operation, the operation of step 201 is repeated at the same scanned location of the same subject.

Step 205: retrieving from the database the subject's image before the acupotome operation according to the instruction of the doctor, displaying simultaneously via the user interface the images before and after the acupotome operation, so as to facilitate the doctor to compare the images before and after the acupotome operation, performing the evaluation of the operation's effectiveness according to the comparing results inputted by the doctor, and storing the evaluation results in the database.

In addition, it is possible for the computer aided apparatus to execute the operation of step 202 after step 204 on the image after the acupotome operation so as to determine the particular feature information after the acupotome operation, and retrieve from the database the particular feature information of the subject before the acupotome operation according to the instruction of the doctor, compare the particular feature information before and after the acupotome operation, perform the evaluation of the operation's effectiveness according to the comparison result, and store the evaluation result in the database and display it to the doctor.

During the evaluation, it can be performed by quantitative comparison, for example, it is possible to compare the diameter of the tumor, perform evaluation of the operation's effectiveness according to the amount of reduction in the diameter, and also perform evaluation of the operation's effectiveness by means of the tumor's level of color depth and so on. The doctor can determine whether to perform a further acupotome operation according to the evaluation result.

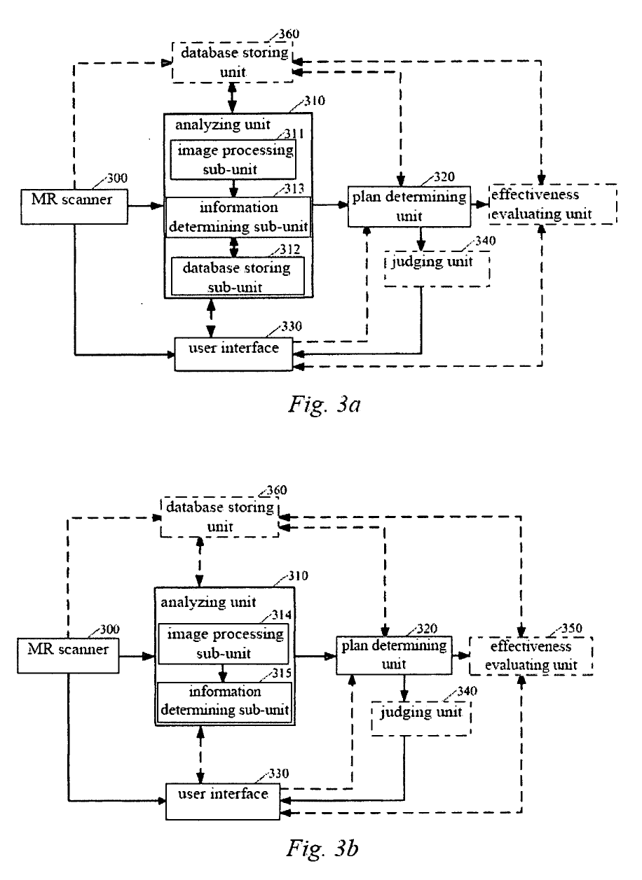

The above is the description of the method provided in an embodiment of the present invention, and the apparatus provided in an embodiment of the present invention will be described below in detail. FIG. 3a is a structural diagram of an apparatus provided according to the embodiments of the present invention, and as shown in FIG. 3a, the apparatus can comprise: a magnetic resonance scanner 300, an analyzing unit 310, a plan determining unit 320 and a user interface 330.

The magnetic resonance scanner 300 is used for acquiring image data of the scanned location by performing a nuclear magnetic resonance scanning on the scanned location of the subject.

The analyzing unit 310 is used for determining the particular feature information with respect to the image by using the image segmentation algorithm.

The plan determining unit 320 is used for determining the position of the acupotome insertion point according to the particular feature information.

The user interface 330 is used for displaying the image formed by the image data, and displaying the position of the acupotome insertion point on the image.

In this case, the analyzing unit 310 can comprise: an image processing sub-unit 311, a database storing sub-unit 312 and an information determining sub-unit 313.

The image processing sub-unit 311 is used for processing the image by using the image segmentation algorithm and providing the processed results to the user interface 330.

The database storing sub-unit 312 is used for storing the pre-set particular feature information corresponding to a variety of processed results.

The information determining sub-unit 313 is used for looking up the particular feature information corresponding to a processed result that is the closest to the processed results in the database storing sub-unit 312, and providing the found particular feature information to the user interface 330.

Here, the user interface 330 can also be used for displaying the processed result provided by the image processing sub-unit 311 and the particular feature information provided by the information determining sub-unit 313.

The structure of the above analyzing unit 310 can be used for accomplishing automatic determination of the particular feature information. In addition, the analyzing unit 310 can also employ another structure for accomplishing semi-automatic determination of the particular feature information, as shown in FIG. 3b. Here, the analyzing unit 310 can comprise an image processing sub-unit 314 and an information determining sub-unit 315.

The image processing sub-unit 314 is used for processing the image by using the image segmentation algorithm and providing the processed result to the user interface 330.

The user interface 330 can also be used for providing a measurement tool to the user, providing the measured result obtained after having performed a measurement with the measure tool by the user to the information determining sub-unit 315, and displaying the processed result provided by the image processing sub-unit 314 and the particular feature information provided by the information determining sub-unit 315.

The information determining sub-unit 315 is used for determining the particular feature information according to the above measured result, and providing the particular feature information to the user interface 330.

If the determination of a surgical operation plan is by way of trigger by the user, then the user interface 330 can also be used for sending a trigger notification to the plan determining unit 320 upon receiving a trigger for determining a surgical operation plan performed by the user via the user interface.

After having received the trigger notification, the plan determining unit 320 determines the position of the acupotome insertion point by using a pre-set program according to the particular feature information.

In addition, if the scanned location before a nuclear magnetic resonance scanning is the one that is marked with a checkpoint in advance, then the apparatus can further comprise: a judging unit 340 for judging whether the distance between the position of the checkpoint and the position of the acupotome insertion point determined by the plan determining unit 320 is within a set range or not, if it is no, then providing the position of the acupotome insertion point to the user interface 330 for displaying, and prompting the user that the marking of the position of the checkpoint is wrong via the user interface 330, and if it is yes, then prompting the user that the marking of the position of checkpoint is correct via the user interface 330.

Furthermore, the apparatus can comprise: an effectiveness evaluating unit 350 for performing an evaluation of the operation's effectiveness according to the comparison result provided by the user interface 330, and providing the evaluation result to the user interface 330.

The user interface 330 is also used for displaying the image before the acupotome operation and the image after the acupotome operation which is acquired by the magnetic resonance scanner 300, receiving the comparison result inputted by the doctor, providing the comparison result to the effectiveness evaluating unit 350, and displaying the evaluation result.

Alternatively, the effectiveness evaluating unit 350 is used for comparing the feature information before an acupotome operation with the feature information after an acupotome operation determined by the analyzing unit 310, performing evaluation of the operation's effectiveness according to the comparison result, and providing the evaluation result to the user interface 330.

The user interface 330 is also used for displaying the evaluation result.

In order to facilitate the storage of a variety of information of the subject, and to facilitate future inquiry and analysis, the apparatus can further comprise: a database storing unit 360 for storing in a database set up for the subject one of the image data, particular feature information, position of the acupotome insertion point and evaluation result or any combination thereof.

The analyzing unit 310 can also be used for acquiring the image data from the database storing unit.

The plan determining unit 320 and the effectiveness evaluating unit 350 are also used for acquiring the particular feature information from the database storing unit 360.

It can be seen from the above description that the method and apparatus provided in at least one embodiment of the present invention can acquire image data of the scanned location by performing a nuclear magnetic resonance scanning on the scanned location of a subject, and display the image formed by the image data via a user interface; determine particular feature information with respect to the image by using an image segmentation algorithm, determine the position of an acupotome insertion point according to said particular feature information, and display the position of the acupotome insertion point on the image. The present invention can provide to a doctor the image information formed after having scanned the scanned location, which image can reflect the ailment condition of the scanned location, determine the particular feature information by further using the image segmentation algorithm, and can determine the position of the acupotome insertion point according to the determined particular feature information, which provide objective evidence that determines the acupotome surgical operation plan for the doctor, which is unlike the prior art which determines the acupotome surgical operation plan only according to some subjective evidence such as the subject's responses upon palpating and the doctor's personal experience, thereby improving the accuracy of acupotome treatment.

In addition, the method and apparatus provided in at least one embodiment of the present invention can also compare the particular feature information before and after the acupotome operation, perform the evaluation of the operation's effectiveness according to the comparison result, and display the evaluation result to the doctor, thereby visually providing to the doctor the treatment effectiveness of the acupotome operation, so as to facilitate the determination of a further surgical operation plan for the doctor. Furthermore, the scanned image, the determined particular feature information, the determined acupotome surgical operation plan and the evaluation result of the operation's effectiveness can be stored in a database set up for the subject to facilitate the future retrieval and analysis.

What are described above are merely example embodiments of the present invention, and are not intended to limit the present invention; any modification, equivalent replacement and improvement, etc. within the spirit and principle of the present invention should be covered in the protective scope of the present invention.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combineable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, computer readable medium and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable medium and is adapted to perform any one of the aforementioned methods when run on a computer device (a device including a processor). Thus, the storage medium or computer readable medium, is adapted to store information and is adapted to interact with a data processing facility or computer device to execute the program of any of the above mentioned embodiments and/or to perform the method of any of the above mentioned embodiments.

The computer readable medium or storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A computer aided method, comprising:
acquiring image data of a scanned location of a subject by performing a nuclear magnetic resonance scan thereon, and displaying an image formed by the acquired image data via a user interface; and
determining particular feature information by using an image segmentation algorithm to the image, determining a position of an acupotome insertion point according to the determined particular feature information, and displaying the determined position of the acupotome insertion point on the image.

2. The method as claimed in claim 1, wherein said determining of the particular feature information by using an image segmentation algorithm to the image further comprises:
processing the image by using the image segmentation algorithm, and displaying a result of said processing on the user interface; and
searching for particular feature information corresponding to the processed result that is the closest to the processed result in a pre-set information database, wherein the information database stores particular feature information corresponding to a variety of processed results.

3. The method as claimed in claim 1, wherein said determining the position of an acupotome insertion point according to said particular feature information in step B comprises:
automatically determining the position of the acupotome insertion point by using a pre-set strategy according to the particular feature information.

4. The method as claimed in claim 1, wherein said scanned location for nuclear magnetic resonance scanning is a location for scanning that is marked with a checkpoint in advance, wherein the position of the checkpoint is displayed in the image formed, and wherein the method further comprises, before displaying the position of the acupotome insertion point on said image,
judging whether the distance between the position of the checkpoint and the position of the acupotome insertion point is within a set range,
if an answer to the judging is no, then continuing the execution of the operation of displaying the position of the acupotome insertion point on the image, and prompting the user that the marking of the position of the checkpoint is wrong, and
if an answer to the judging is yes, then prompting the user that the marking of the position of the checkpoint is correct.

5. The method as claimed in claim 1, further comprising:
executing said acquiring again on the scanned location of the subject after having performed an acupotome operation on the subject; and
displaying simultaneously the image before the acupotome operation and the image after the acupotome operation via the user interface, evaluating the operation's effectiveness by using a comparison result inputted by a doctor, and displaying the evaluation result via said user interface.

6. The method as claimed in claim 5, wherein at least one of said image data, the particular feature information, the position of the acupotome insertion point and the evaluation result is stored in a database set up for said subject.

7. A computer aided apparatus, comprising:
a magnetic resonance scanner to acquire image data of a scanned location of a subject by performing a nuclear magnetic resonance scanning thereon;
an analyzing unit to determine particular feature information by using an image segmentation algorithm to an image formed by said acquired image data;
a plan determining unit to determine a position of an acupotome insertion point according to the determined particular feature information; and
a user interface to display the image formed by the image data, and to display the determined position of the acupotome insertion point on the image.

8. The apparatus as claimed in claim 7, wherein said analyzing unit comprises:
an image processing sub-unit to process the image by using an image segmentation algorithm, and to provide the processed result to said user interface;
a database storing sub-unit to store the pre-set particular feature information corresponding to a variety of processed results; and
an information determining sub-unit to search particular feature information corresponding to a processed result that is the closest to said processed result in said database storing sub-unit, and to provide the particular feature information found to said user interface, and wherein said user interface is also used to display said processed result provided by said image processing sub-unit and said particular feature information provided by said information determining sub-unit.

9. The apparatus as claimed in claim 7, wherein said analyzing unit comprises:
an image processing sub-unit to process said image by using an image segmentation algorithm, and to provide said processed result to said user interface, wherein said user interface is also used for providing a measurement tool to a user, and for providing the measured result obtained by the user after having performed a measurement with the measurement tool to an information determining sub-unit, and for displaying said processed result provided by said image processing sub-unit and said particular feature information provided by said information determining sub-unit; and
said information determining sub-unit is also used for determining the particular feature information according to said measured result, and for providing said particular feature information to said user interface.

10. The apparatus as claimed in claim 7, wherein said user interface is also used for sending a trigger notification to said plan determining unit upon receiving a trigger for determining a surgical operation plan performed by a user via the user interface; and
after having received said trigger notification, said plan determining unit determines the position of said acupotome insertion point by using a pre-set strategy according to said particular feature information.

11. The apparatus as claimed in claim 7, wherein said scanned location by nuclear magnetic resonance scanning is a location for scanning that is marked with a checkpoint in advance; the apparatus further comprising:
a judging unit to judge whether the distance between the position of said checkpoint and the position of the acupotome insertion point determined by said plan determining unit is within a pre-set range, and if the distance is judged not to be within the pre-set range, then providing the position of the acupotome insertion point to said user interface for displaying, and prompting the user via the user interface that the marking of the position of the checkpoint is wrong; if the distance is judged to be within the pre-set range, then prompting the user via the user interface that the marking of the position of the checkpoint is correct.

12. The apparatus as claimed in claim 7, further comprising:
an effectiveness evaluating unit to perform evaluation of an operation's effectiveness according to the comparison result provided by said user interface, and to provide the evaluation result to said user interface; wherein
said user interface is also used to display the image before the acupotome operation and the image after the acupotome operation which are acquired by said magnetic resonance scanner, to receive the comparison result inputted by a doctor, to provide the comparison result to said effectiveness evaluating unit, and to display said evaluation result.

13. The apparatus as claimed in claim 7, further comprising:
an effectiveness evaluating unit to compare the feature information before the acupotome operation with the feature information after the acupotome operation determined by said analyzing unit, to evaluate the operation's effectiveness according to said comparison result, and to provide the evaluation result to said user interface, wherein said user interface is also used for displaying said evaluation result.

14. The apparatus as claimed in claim 12, further comprising:
a database storing unit to store one of said image data, the particular feature information, the position of the acupotome insertion point and the evaluation result or any combination thereof in a database set up for said subject, wherein said analyzing unit is also used for acquiring the image data from said database storing unit; and
said plan determining unit and effectiveness evaluating unit are also used for acquiring the particular feature information from said database storing unit.

15. The method as claimed in claim 1, wherein said determining of the particular feature information by using an image segmentation algorithm to the image further comprises:
providing to a user a measurement tool on the user interface; and
determining the particular feature information according to a measured result obtained by the user after having performed a measurement with the measurement tool.

16. The method as claimed in claim 1, wherein said determining the position of an acupotome insertion point according to said particular feature information comprises:
determining, upon receiving a trigger for determining a surgical operation plan performed by the user via the user interface, the position of the acupotome insertion point by using a pre-set strategy according to the particular feature information.

17. The method as claimed in claim 1, further comprising:
continuing to execute the determining of particular feature information by using an image segmentation algorithm to the image after having executed said acquiring again on the scanned location of the subject;
comparing the particular feature information obtained before the acupotome operation with the particular feature information obtained after the acupotome operation;
evaluating the operation's effectiveness according to the comparison result; and
displaying the evaluation result via the user interface.

18. The method as claimed in claim 5, wherein the particular surgical operation plan information determined by a user according to the particular feature information is stored in the database set up for the subject, and wherein said detailed surgical operation plan information comprises at least one of a direction of the acupotome insertion and a depth of the acupotome insertion.

19. A computer readable medium including program segments for, when executed on a computer device, causing the computer device to implement the method of claim 1.

20. The apparatus as claimed in claim 10, wherein said scanned location by nuclear magnetic resonance scanning is a location for scanning that is marked with a checkpoint in advance; the apparatus further comprising:
a judging unit to judge whether the distance between the position of said checkpoint and the position of the acupotome insertion point determined by said plan determining unit is within a pre-set range, and if the distance is judged not to be within the pre-set range, then providing the position of the acupotome insertion point to said user interface for displaying, and prompting the user via the user interface that the marking of the position of the checkpoint is wrong; if the distance is judged to be within the pre-set range, then prompting the user via the user interface that the marking of the position of the checkpoint is correct.

21. The apparatus as claimed in claim 13, further comprising:
a database storing unit to store one of said image data, the particular feature information, the position of the acupotome insertion point and the evaluation result or any combination thereof in a database set up for said subject, wherein said analyzing unit is also used for acquiring the image data from said database storing unit; and
said plan determining unit and effectiveness evaluating unit are also used for acquiring the particular feature information from said database storing unit.
